(19) United States
(12) Patent Application Publication
(10) Pub. No.: US 2022/0183415 A1
(43) Pub. Date: Jun. 16, 2022

(54) SMART OQAL

(71) Applicants: KING ABDULLAH UNIVERSITY OF SCIENCE AND TECHNOLOGY, Thuwal (SA); KING FAHD UNIVERSITY OF PETROLEUM AND MINERALS, Dhahran (SA)

(72) Inventors: Ali Hussein MUQAIBEL, Dhahran (SA); Tareq Yousef AL-NAFFOURI, Thuwal (SA); Mohanad AHMED, Thuwal (SA)

(21) Appl. No.: 17/603,045

(22) PCT Filed: Apr. 13, 2020
(86) PCT No.: PCT/IB2020/053476

§ 371 Date: Oct. 12, 2021

Related U.S. Application Data
(60) Provisional application No. 62/835,860, filed on Apr. 18, 2019.

(51) Int. Cl. A41G 5/00; G06F 3/01; G06F 1/16; H04W 4/90

(52) U.S. Cl. CPC A41G 5/0093; H04W 4/90; G06F 1/163; G06F 3/012

(57) ABSTRACT

A smart oqal includes a first loop; a second loop; and a smart pack attached to at least one of the first and second loops. The smart pack is located to not be visible when the oqal is worn on a head of a user, and the smart pack includes at least one sensor for detecting a parameter associated with the head of the user.

### CROSS-REFERENCE TO RELATED APPLICATIONS

This application claims priority to U.S. Provisional Patent Application No. 62/835,860, filed on Apr. 18, 2019, entitled “SMART OQAL/HAT CAPABLE OF HEAD POSTURE SENSING,” the disclosure of which is incorporated herein by reference in its entirety.

### BACKGROUND

### Technical Field

Embodiments of the subject matter disclosed herein generally relate to monitoring one or more parameters related to a human, and more specifically, placing one or more monitoring sensors on an oqal for observing the wearer's behavior and/or associated parameters.

### Discussion of the Background

Recently, with the advance of miniature electronics, cameras and processing devices, a myriad of wearable devices have been brought to the market for helping the wearer to receive enhanced visual information, in addition to other types of information. This enhanced visual information is projected by a head-mounted display or heads-up display that is worn by the user. The enhanced visual information may include pictures, videos, written information associated with an object to which the user is looking at or any other type of information.

To be able to see this information, as illustrated in FIG. 1, the user 102 wears a head-mounted display 110, which is supported by a band 112 that provides the desired fit of the display on the user's head (this configuration corresponds to FIG. 6 of U.S. Pat. No. 9,195,067 patent). Band 112 is configured such that when properly worn by the user, the display 110 can be positioned adjacent to the user's eye for making an image presented thereon viewable by the user. The band may receive an input from the user via a touch-based input 170 that is accessible to the user and is configured to receive a touch input from the user to execute a control function of the device or a function of another electronic device that is connected or in communication with device.

Additional input structures can be added to band 112, as for example, a camera 126 and a sensor 128. The camera 126 can be used to capture an image or video at the user's discretion. The camera 126 can also be used by the device to obtain an image of the user's view of his or her environment to use in implementing augmented reality functionality. The sensor 128 can be, for example a light sensor that can be used by firmware or software associated with the camera 126.

Similar wearable devices that include a screen and a camera have been developed by other manufacturers and commercialized. However, these products have failed to penetrate the market because they not only look unusual, but mostly the users complain about the difficulty to watch with one eye the small screen 110 and with the other eye the ambient, and there are studies that indicate that the brain has a hard time to integrate these separate images into a single perception of the ambient.

Therefore, there is a need for another type of wearable devices that are still capable of collecting information about the user, but that can present this information to the user without straining the visual capabilities of the user and/or without generating uncomfortable feelings.

### SUMMARY

According to an embodiment, there is a smart oqal that includes a first loop, a second loop, and a smart pack attached to at least one of the first and second loops. The smart pack is located to not be visible when the oqal is worn on a head of a user, and the smart pack includes at least one sensor for detecting a parameter associated with the head of the user.

According to another embodiment, there is an article of clothing that is worn on a head of a user, the article of clothing including a scarf that is worn on the head of the user, and an oqal that is placed over the scarf to maintain the scarf fixed relative to the head of the user. The oqal includes a first loop and a second loop, and a smart pack attached to at least one of the first and second loops. The smart pack is located to not be visible when the oqal is worn on the head of the user, and the smart pack includes at least one sensor for detecting a parameter associated with the head of the user.

According to still another embodiment, there is a method of monitoring a parameter of a user. The method includes placing a scarf on a head of the user, fixing the scarf to the head of the user with an oqal, monitoring the parameter of the user with at least one sensor that is attached to the oqal, and transmitting with a transceiver attached to the oqal, data collected by the at least one sensor to a mobile phone of the user.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 illustrates a head mounted device that projects visual information to the user of the device;

FIG. 2 shows an oqal that fixes a scarf to the head of the user;

FIG. 3 shows a smart oqal that includes a smart pack that collects data associated with the user;

FIGS. 4A to 4C illustrate the smart pack, its electronic components, and its mechanical components that are used to attach it to the oqal;

FIG. 5 illustrates a smart oqal that has the smart pack integrated inside the oqal;

FIG. 6 illustrates the smart pack that is configured to fully fit inside the smart oqal;

FIG. 7 illustrates the smart pack integrated inside the smart oqal;

FIG. 8 is a flowchart of a method for collecting data with the smart pack attached to the oqal and transmitting it to a mobile device used by the user;

FIG. 9 is a flowchart of a method for wearing a smart oqal and receiving information on a mobile device from the smart oqal;

FIG. 10A illustrates the smart pack implemented into a hat; and

FIG. 10B illustrates the smart pack implemented into a hair clip.

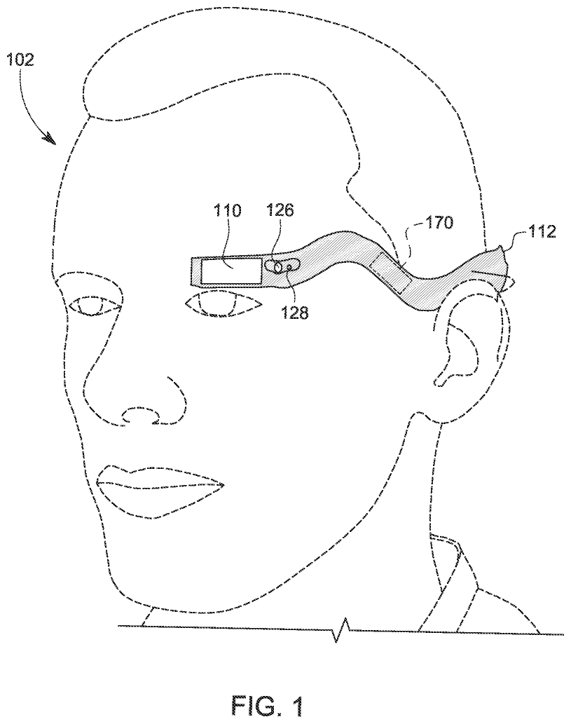

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to an oqal that has two loops. However, the embodiments discussed herein can be applied to an oqal that has more or less loops, to loops that are concentric, or to loops that are shaped to have a hexagonal shape, or to an oqal that has a single loop or even to another article of clothing.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, an Arabian headwear, which is used to fix the Gotra, a scarf worn on the head, is provided with one or more sensors for collecting data, a power supply, a processor and a transceiver for exchanging the data with another device, for example, a mobile device. The Arabian headwear, known also under the name of oqal, igal, agal, or eqal is traditionally worn in the Arab countries for fixing the scarf to the head. Thus, the oqal is always placed on the head. By adding one or more sensors to the oqal, enables the user of the oqal to collect data related not only to a position of the head, but also related to various body parameters, e.g., temperature, sun exposure, blood pressure, etc., as now discussed. This data is then transferred to the mobile device, where it may be processed and presented to the user on the display of the mobile device.

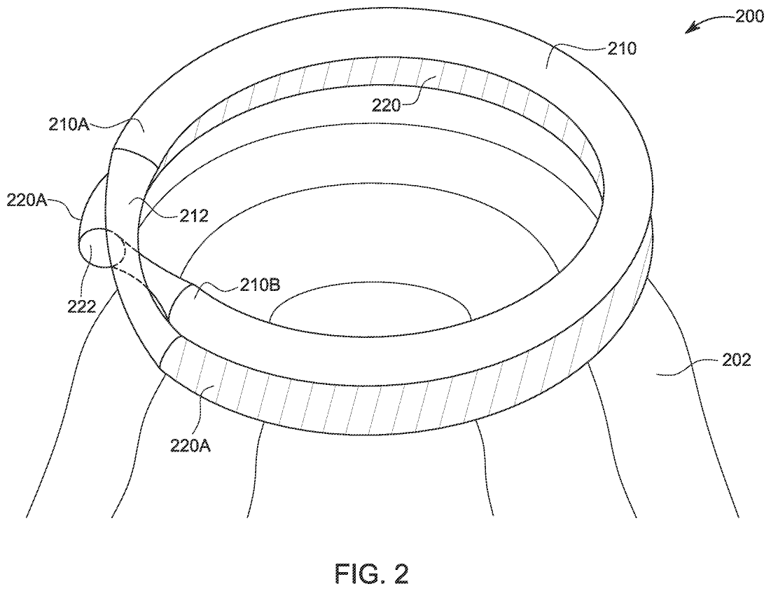

An oqal 200 is shown in FIG. 2 having first and second concentric loops 210 and 220, whose ends are connected to each other. The first and second loops 210 and 220 are placed over a scarf 202, to fix the scarf relative to the head of the user (not shown). A first end 210A of the first loop 210 is connected to a first end 220A of the second loop 220 and a second end 210B of the first loop 210 is connected to a second end 220B of the second loop 220. The first and second loops are more or less circular, to fit the head of the user. The connection parts 212 and 222 between the ends of the two loops cross each other as shown in the figure. The oqal 200 is traditionally made from a fabric material.

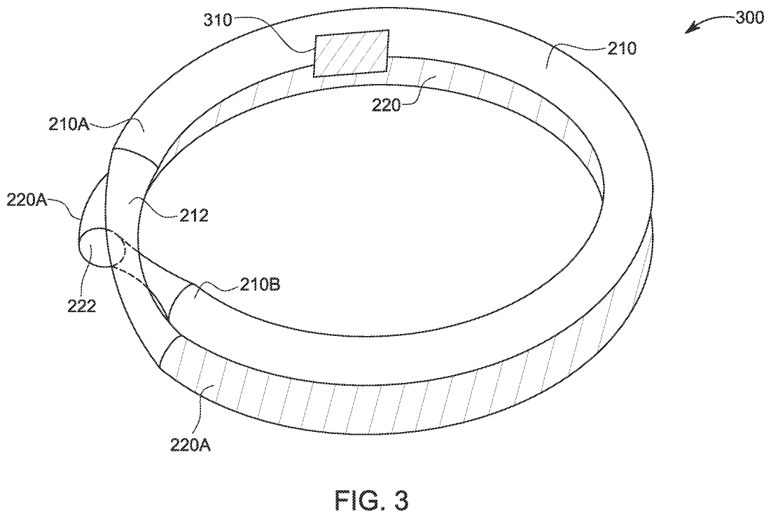

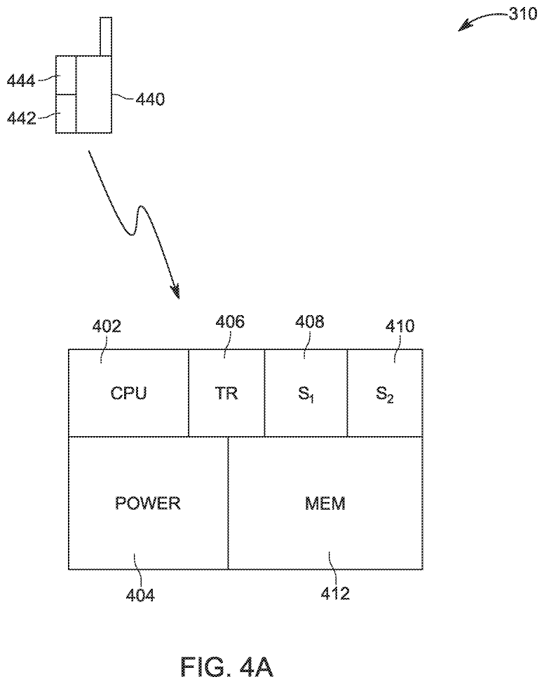

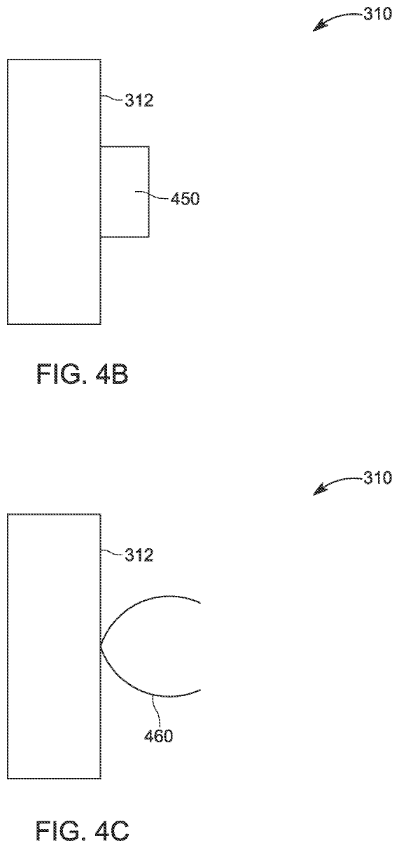

According to the embodiment illustrated in FIG. 3, a smart pack 310 is attached to the oqal 300. The smart pack 310 is attached in this embodiment to an interior part of the oqal, so that a person other than the user cannot see the smart pack. The smart pack 310, which is shown in more detail in FIGS. 4A to 4C, includes a processor 402, a power pack 404, a transceiver 406, and a first sensor 408. FIG. 4A shows the smart pack 310 having also a second sensor 410. However, the second sensor is optional. In one application, the smart pack 310 has plural sensors. The transceiver 406 is configured to receive and transmit information to a remote device 440, for example, a mobile phone. Any other device may be used as the remote device as long as this device has a processor and wireless communication means. The transceiver 406 can transmit data from the CPU 402 to the remote device 440 and can receive instructions from the remote device. For example, in one embodiment, the data collected by the various sensors in the smart pack 310 are transmitted by the processor 402, through the transceiver 406, to the remote device 440. The remote device 440 may use its own processing capabilities for processing the collected data and for displaying the data. In this way, the user does not have to strain her or his eyes to see the collected data. In fact, the user may chose when to see the data and on which kind of device or screen to see the data. Note that the transceiver 406 may use any protocol for exchanging data with the remote device 440, for example, Wi-Fi, Bluetooth, infrared, RF, etc. Any encoding method may be used to encode the data, if desired. Also, any modulation technique may be used for transmitting the data.

The processor 402 is also connected to a memory 412 for storing data and/or instructions. The data collected from the first sensor 408 (the same is true for the other sensors is they are present) may be stored in the memory 412 before being transmitted to the remote device 440. The processor 402 may be a more complex integrated circuit that may partially or totally process the collected data. The processor 402, the memory 412, the transceiver 406 and the one or more sensors 408 are supplied with power from the power pack 404. The power pack 404 may include a battery, a solar cell, a fuel cell, or any known device for generating electricity.

In one embodiment, the first sensor 408 is a motion sensor that determines an acceleration or speed or displacement of the oqal, and implicitly of the head of the user. For example, the motion sensor may be an accelerometer (one, two or three axial), geophone, etc. In another application, the first sensor is a gyroscope, so that an orientation of the head of the user can be monitored. In still another application, the first sensor 408 is a motion sensor and the second sensor 410 is the gyroscope. The first sensor may include all or only some of the sensors discussed above. In one application, the first sensor may include other sensors, as for example, a temperature sensor, a pressure sensor, and/or a light intensity sensor.

Regardless of the specific implementation of the sensors, the information collected by the processor 402 may be used to continuously monitor the position of the user's head. This processing may take place at the local processor 402, or at the global processor 442 of the remote device 440. In one application, the processing of the data from the sensors is shared between the local processor 402 and the global processor 442.

The result of the processed data (e.g., head posture) may be displayed on a display 444 of the remote device 440. Note that in this embodiment, the smart oqal 300 does not have any display means. The remote device 440, when carried by the user, for example, in his pocket, may warn the user about her or his posture. For example, if the processor determines that the head is tilted too much forward, for a long period of time, the remote device may issue a visual or auditive alarm for the user to correct its posture.

In another application, the processor may be programmed to determine if the head of the user is showing signs of sleeping while driving. In this instance, based on the data collected from the one or more sensors 408, the processor 402 or 442 or both can determine that the user is close to sleeping or is already sleeping and can alert the user, by emitting a visual or acoustic or both signals to wake up the driver. In one application, the remote device communicates with a global controller of the car or the smart oqal is programmed to directly communicate with the global controller of the car and if the processor determines that the driver is asleep, it can instruct the car to automatically brake to prevent an accident. In one application, the sleep alarms generated by the processors are sent to the user only if the remote device 440 validates that indeed the user is driving a car, which can be implemented by determining that a speed of the user relative to ground is larger than a certain value, for example 30 km/h. In this way, the remote device 440 confirms that the user is driving a car and allows the alarm to be sent to the user.

Other sensors may be implemented in the smart pack 310. For example, the oqal is traditionally worn in the Arab countries, where the amount of sun to which the wearer is exposed is high. Thus, one of the sensors 408 or 410 may be photodetector or similar device that measures the light intensity. The processor 402 may be programmed to calculate the amount of solar radiation that is received by the head of the user and if the amount is larger than a given threshold, the processor 402 may send a warning to the processor 442 of the remote device 440, to alert the user that he or she needs to take cover to reduce the risk of sun exposure.

In another embodiment, the processor 402 may be configured to enter a sleep state when no instructions from the remote device 440 is received, to conserve power. During the sleep state, the processor 402 is configured to switch off the power supply to the sensors. Only the transceiver 406 is powered during the sleep state. During this state, the transceiver 406 is still functional and can receive information. As soon as the transceiver 406 detects an incoming signal from the remote device 440, the transceiver 406 sends the signal to the processor 402 and wakes it up. Then, the processor 402 reads the incoming signal. If a request has been received from the user to monitor her or his posture or sun exposure or any other parameter, the processor instructs the power pack 404 to provide electrical power to the sensors so that data can be collected. This process continues until the user sends a sleep instruction to the processor 402, via the remote device 440.

In still another embodiment, it is possible that one of the sensors is a temperature sensor and the processor, after processing the collected data, informs the user, via the remote device 440, about the ambient temperature, especially around the user's head, to prevent various medical conditions associated with extended sun exposure.

To attach the smart pack 310 to the oqal 300 as shown in FIG. 3, in one embodiment, which is illustrated in FIG. 4B, a hook-and-loop fastener 450 (also known as a Velcro band) is attached to a housing 312 of the smart pack 310. A similar fastener is sewed to the oqal and then, the smart pack is easily attached to the oqal. Other fasteners may be used to attach the smart pack to the oqal. For example, FIG. 4C shows a clamp 460 that is attached to the housing 312 and the clamp is configured to be placed around one or both loops 210 and 220 of the oqal 300. While FIGS. 4A to 4C show all the components of the smart pack 310 being placed in a single housing 312, one skilled in the art would understand that the smart pack may have multiple housings, each configured to accommodate a subset of the electronic components shown in FIG. 4A.

Returning to FIG. 3, the smart pack 310 is attached to an interior of the loops 210 and/or 220 so that it is not visible by another person. In other words, the smart pack is attached to the oqal in such a way that another person that is facing the user of the oqal cannot see the smart pack. In this way, the smart oqal still complies with the tradition.

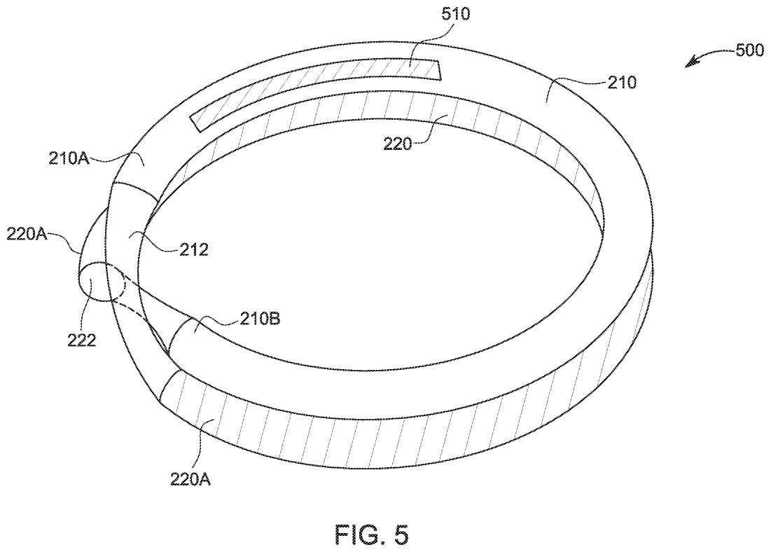

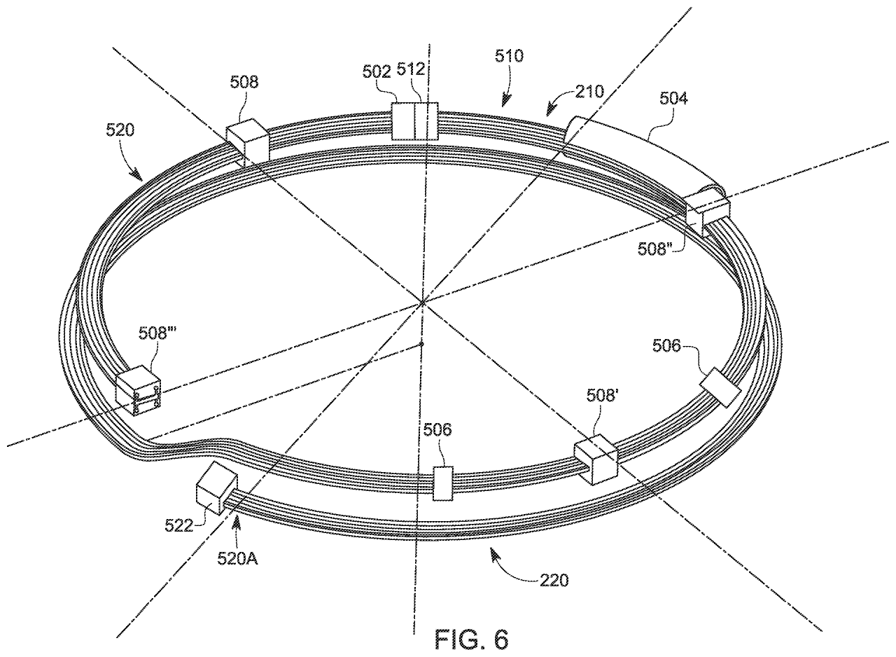

In still another embodiment illustrated in FIG. 5, the oqal 500 has a smart pack 510 that is fully embedded into the body of the oqal so that the smart pack cannot be seen even if the oqal has been removed from the head of the user. In other words, as illustrated in FIG. 5, the smart pack 510 is completely within one or both loops 210 and 220. FIG. 5 shows the smart pack 510 being placed inside one loop 210. However, the smart pack 510 may extend in both loops 210 and 220.

In this respect, FIG. 6 shows a smart pack 510 that is designed to sit inside both loops 210 and 220. Smart pack 510 has a bus 520 that connects the various electrical components to each other. FIG. 6 shows a processor 502, power pack 504, transceiver 506, plural sensors 508 to 508′″, and memory 512. Each of these elements is attached to the bus 520 and can communicate with each other or can receive power from the power pack. These elements may be distributed along the bus 520 in any configuration, for example, packed together or spaced apart. In one application, the sensors 508 to 508′″ may be identical and equally distributed along the bus 520 for more accurately capturing a movement of the head. As previously discussed, other type of sensors may be used.

In one application, one end 520A of the bus 520 ends with a plug 522. The plug, which may be located in the second loop 220, may be configured to connect to a charger (not shown), so that the battery pack 504 may be charged overnight. The plug may also be configured to directly connect to the remote device 440 so that the data stored in the memory 512 may be transferred to the remote device. In still another application, the connector 522 may be used to transfer commands or instructions from the remote device to the processor 502. Note that this wired connection of the oqal to the remote device 440 may occur in addition to the wireless connection that is established trough the transceiver 406.

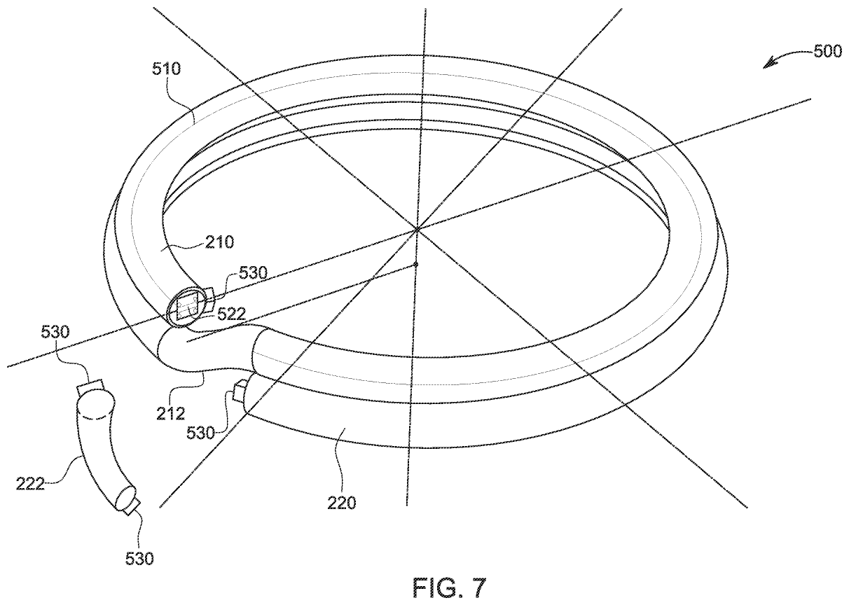

The smart pack 510 shown in FIG. 6 may be fully embedded into the first and second loops 210 and 220, as shown in FIG. 7. For this embodiment, the smart pack 510 is not visible from outside, even if the oqal is removed from the head of the user. FIG. 7 shows the connector 522 being visible because the connection part 222 has been temporarily removed. The connection part 222 can be attached to the ends of the first and second loops 210 and 220, when necessary, for example, with a hook-and-loop fastener 530, which is similar to fastener 450. Other methods for connecting the ends of the first and second loops may be used.

The smart pack 510 may be similar to the smart pack 310 discussed above, and thus, its description is not repeated herein. The same functionalities as discussed above for the smart pack 310 may be implemented in the smart pack 510. In one embodiment, the remote device 440 runs an application that allows the user of the oqal to interact with the smart pack 510. For example, the user may be allowed to introduce a range of head motions that would not trigger an alarm by the processor 502 and/or 442. Also, the user may interact with the processor 502 in the smart pack 510 and instruct the processor to issue an alarm if the light intensity measured by a light sensor connected to the processor 502 is over a given threshold, over a given time interval. The same may happen for the other sensors that may be fitted to the smart pack.

Although the smart packs 310 or 510 do not contain any physical interface that allows the user to interact with it, e.g., to have a tapping surface or a button, in one embodiment it is possible to add such capability to the smart packs. However, a goal of the smart pack 310 or 510 is to not have any manual interaction with the user and also to not present any image or visual information directly to the user. All the information collected by the smart pack is presented to the user via the remote device 440 and the user interacts with the smart pack of the oqal only through the remote device.

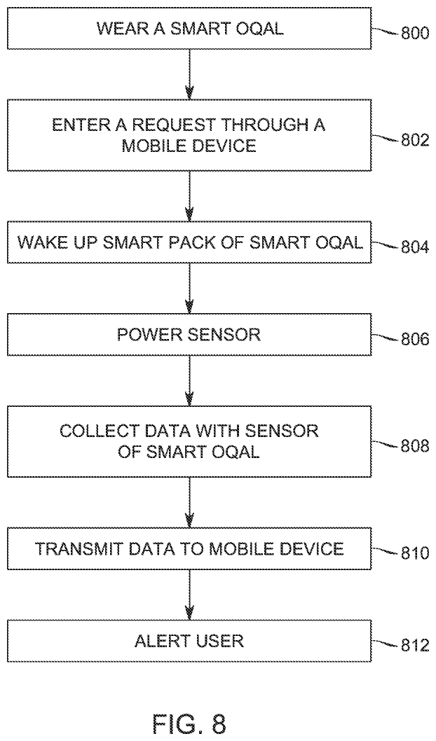

Thus, according to a method that is illustrated in FIG. 8, a user wears in step 800 an oqal to fix a scarf that is provided over the head of the user. Note that the oqal is designed to have one or more loops that fit on the head of the user. A smart pack may be attached to one of the loops, as shown in FIG. 3 or may be fully embedded inside one or more of the loops, as shown in FIG. 5. In step 802, the user enters a requirement through the remote device 440, for example, for monitoring its posture, or sun exposure, or for monitoring any other parameter. The remote device 440 wakes up in step 804 the smart pack, and the local processor 402 of the smart pack instructs the power pack to provide power, in step 806, to the one or more sensors present in the smart pack. Note that in this embodiment there is no display or an interface so that the user can directly send instructions to the smart pack.

In step 808, the one or more sensors collect data and transmit it to the local processor 402. In step 810, the local processor 402 transmits information, through the transceiver 406, to the global processor 442 of the remote device 440 and the remote device 440 alerts in step 812 the user if the posture is not correct, or if the sun exposure is too high. Other applications of the sensors may be implemented as for example, head temperature monitoring, number of calories used for various activities, sign recognition and control, etc. About the sign recognition and control, it is possible that at least one sensor is an accelerometer, which can identify when the head is moved backward and forward, or left and right, and these motions may be associated with various controls of the remote device, for example, manipulating the volume of the device. Those skilled in the art would know to implement other applications based on the sensors of the oqal.

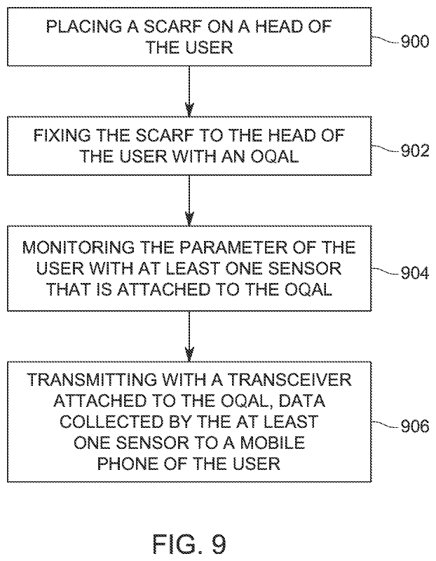

According to an embodiment illustrated in FIG. 9, there is a method of monitoring a parameter of a user that includes a step 900 of placing a scarf on a head of the user, a step 902 of fixing the scarf to the head of the user with an oqal, a step 904 of monitoring the parameter of the user with at least one sensor that is attached to the oqal, and a step 906 of transmitting with a transceiver attached to the oqal, data collected by the at least one sensor to a mobile phone of the user. In one application, the oqal includes a first loop and a second loop that is concentric to the first loop, and a smart pack attached to the at least one of the first and second loops. The smart pack is located to not be visible when the oqal is worn on the head of the user, and the smart pack includes the least one sensor and the transceiver. The method may further include a step of exchanging information, in a wireless manner, between the transceiver and the mobile phone of the user when the measured parameter is larger than a given threshold. The parameter is a posture of the head or a light intensity.

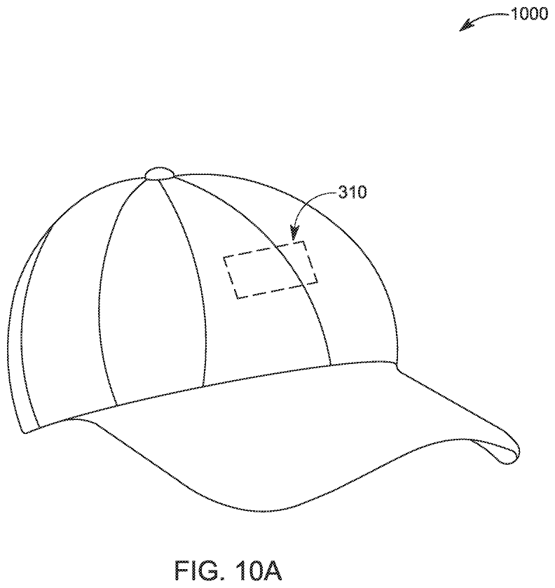

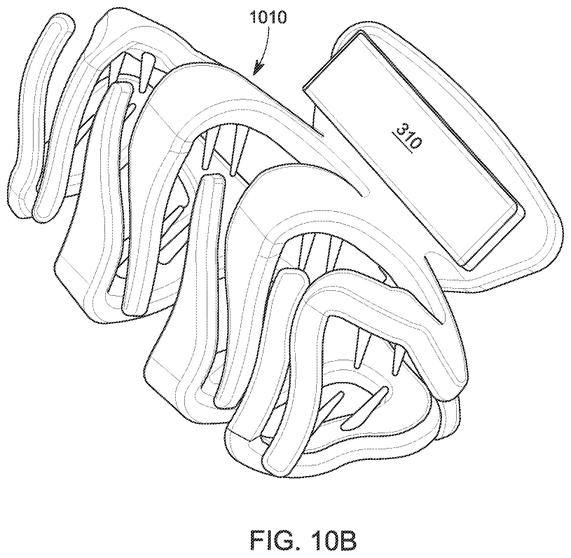

The smart pack 310 shown in FIG. 3 has been discussed with regard to an oqal. However, the smart pack 310 may also be attached to a hat 1000 as shown in FIG. 10A, or to a hair clip 1010 as shown in FIG. 10B. Those skilled in the art would understand that the smart pack may be integrated in other articles of clothing, headwear, or hair ornaments.

The disclosed embodiments provide a smart oqal and methods for monitoring a posture of the head of the user of the oqal or for monitoring various parameters associated with the user of the oqal. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A smart oqal comprising:
a first loop;
a second loop; and
a smart pack attached to at least one of the first and second loops,
wherein the smart pack is located to not be visible when the oqal is worn on a head of a user, and
wherein the smart pack includes at least one sensor for detecting a parameter associated with the head of the user.

2. The smart oqal of claim 1, wherein the smart pack further comprises:
a processor;
a transceiver; and
a power pack that provides electrical power to the processor, the at least one sensor, and the transceiver.

3. The smart oqal of claim 2, wherein the transceiver is configured to exchange information in a wireless manner with a mobile phone.

4. The smart oqal of claim 3, wherein the at least one sensor is an accelerometer that detects a posture of the head of the user.

5. The smart oqal of claim 4, wherein the processor is configured to send an alert to the mobile phone when a posture of the user is beyond a limit.

6. The smart oqal of claim 3, wherein the at least one sensor is a light intensity sensor.

7. The smart oqal of claim 6, wherein the processor is configured to send an alert to the mobile phone when a measured light intensity is above a given threshold.

8. The smart oqal of claim 1, wherein the smart pack attaches with a fastener to the first loop and the first and second loops are made of fabric.

9. The smart oqal of claim 1, wherein the smart pack is fully embedded into at least one of the first and second loops.

10. The smart oqal of claim 1, wherein the smart pack extends in both the first and the second loops.

11. The smart oqal of claim 1, wherein the smart pack includes a bus that extends along at least one of the first and second loops and electrically connects to each other a processor, a transceiver, the at least one sensor, a power pack, and a memory.

12. The smart oqal of claim 1, wherein the at least one sensor includes an accelerometer, a gyroscope and a light intensity sensor.

13. The smart oqal of claim 1, wherein the smart pack does not include a display or an interface that is directly accessed by the user.

14. The smart oqal of claim 1, wherein the first and second loops are concentric, or the first and second loops are shaped to have a hexagonal shape, or the first and second loops form a single loop.

15. An article of clothing that is worn on a head of a user, the article of clothing including:
a scarf that is worn on the head of the user; and
an oqal that is placed over the scarf to maintain the scarf fixed relative to the head of the user,
wherein the oqal includes,
a first loop and a second loop, and
a smart pack attached to at least one of the first and second loops,
wherein the smart pack is located to not be visible when the oqal is worn on the head of the user, and
wherein the smart pack includes at least one sensor for detecting a parameter associated with the head of the user.

16. The article of clothing of claim 15, wherein the smart pack further includes:
a processor;
a transceiver; and
a power pack that provides electrical power to the processor, the at least one sensor, and the transceiver.

17. The article of clothing of claim 16, wherein the transceiver is configured to exchange information in a wireless manner with a mobile phone of the user.

18. A method of monitoring a parameter of a user, wherein the method comprises:
placing a scarf on a head of the user;
fixing the scarf to the head of the user with an oqal;
monitoring the parameter of the user with at least one sensor that is attached to the oqal; and
transmitting with a transceiver attached to the oqal, data collected by the at least one sensor to a mobile phone of the user.

19. The method of claim 18, wherein the oqal includes a first loop and a second loop, and a smart pack attached to at least one of the first and second loops,
wherein the smart pack is located to not be visible when the oqal is worn on the head of the user, and
wherein the smart pack includes the least one sensor and the transceiver.

20. The method of claim 18, further comprising:
exchanging information, in a wireless manner, between the transceiver and the mobile phone of the user when the measured parameter is larger than a given threshold.

21. The method of claim 18, wherein the parameter is a posture of the head or a light intensity.
